(19) United States
(12) Patent Application Publication
(10) Pub. No.: US 2006/0069076 A1
(43) Pub. Date: Mar. 30, 2006

(54) LXR/RXR-related methods and compositions

(76) Inventors: Yu Sun, River Edge, NJ (US); Tae-Wan Kim, Old Tappan, NJ (US); Alan Tall, Cresskill, NJ (US)

Correspondence Address: COOPER & DUNHAM, LLP, 1185 AVENUE OF THE AMERICAS, NEW YORK, NY, 10036, US

(21) Appl. No.: 11/172,317

(22) Filed: Jun. 29, 2005

Related U.S. Application Data
(60) Provisional application No. 60/584,752, filed on Jul. 1, 2004.

(51) Int. Cl. A61K 31/57; A61K 31/203

(52) U.S. Cl. 514/178; 514/559

(57) ABSTRACT

This invention provides methods for decreasing the amount of Aβ peptide produced by a neuronal cell comprising contacting the cell with an agent that, when in contact with the neuronal cell, causes activation of the cell's Liver X Receptor (LXR) and/or Retinoid X Receptor (RXR). This invention also provides for related therapeutic and prophylactic methods. Finally, this invention provides related articles of manufacture.

This application claims the benefit of U.S. Provisional Application No. 60/584,752, filed Jul. 1, 2004, the contents of which are incorporated hereby by reference into the subject application.

The invention disclosed herein was made with government support under National Institutes of Health Grant Nos. HL 22682 and 54591, and National Institutes of Health Grant No. AG 18026. Accordingly, the U.S. Government has certain rights in this invention.

Throughout this application, various publications are referenced by author and date. Full citations for these publications may be found listed alphabetically at the end of the specification immediately preceding the claims. The disclosures of these publications in their entireties are hereby incorporated by reference in order to more fully describe the state of the art.

### BACKGROUND OF THE INVENTION

The deposition of plaques of amyloid β peptide (“Aβ”) in the brain is the hallmark of Alzheimer's disease (“AD”). The generation of Aβ requires sequential cleavage of the type I integral membrane amyloid precursor protein (“APP”) by β- and then γ-secretase. β-Secretase cleaves APP extracellularly, leaving a 99-residue C-terminal fragment (“C99”) that remains membrane-bound. γ-Secretase then mediates an intramembranous cleavage, yielding the Aβ peptide (for review see Refs. 1 and 2). An alternative initial cleavage of APP by α-secretase precludes subsequent Aβ formation. Simons and Ehehalt (3) have proposed that the initial cleavage of APP by β-secretase occurs in cholesterol-rich liquid ordered domains of the plasma membrane known as “rafts.” The basis of this hypothesis was the finding that depletion of cellular cholesterol by cyclodextrin treatment decreases raft formation and markedly reduces Aβ formation (4). Interestingly, presenilins, important components of γ-secretase, have also been found in cholesterol-rich domains (5).

Cellular lipid homeostasis is controlled by sterol regulator element-binding proteins, transcription factors regulating cholesterol and fatty acid synthesis pathways, and by liver X receptors (“LXRs”), oxysterol-activated nuclear receptors that induce a battery of genes involved in cellular lipid efflux and transport (6, 7). The two forms of LXR, α and β, are both expressed in the brain. LXRβ is broadly expressed in the developing and adult brain and is present in both neurons and glial cells (8). Recent studies show an essential role for LXRs in brain structure and function as aging LXRα/β knockout mice develop cellular lipid inclusions, abnormalities of the choroid plexus, and closure of the lateral ventricles (8). Although this pathology is different from that of AD, LXRs could potentially have a role in modulating the course of chronic neurodegenerative diseases.

An important target of LXRs is the ATP-binding cassette transporter A1 (“ABCA1”) (9). ABCA1, the defective molecule in Tangier disease, mediates efflux of cellular phospholipids and cholesterol to lipid-poor apolipoprotein, including apolipoprotein A-I (apoA-I) and apoE, which are present in the cerebrospinal fluid (10). Treatment of mice with LXR agonists resulted in increased expression of LXR target genes in the brain, especially ABCA1 (8), and LXR activation induces lipid efflux from glial cells (11).

### SUMMARY OF THE INVENTION

This invention provides a method for decreasing the amount of Aβ peptide produced by a neuronal cell comprising contacting the cell with an agent that, when in contact with the cell, causes activation of the cell's Liver X Receptor (LXR) and/or Retinoid X Receptor (RXR).

This invention further provides a method for treating a subject afflicted with Alzheimer's disease comprising administering to the subject a therapeutically effective amount of an agent that, when in contact with a neuronal cell, causes activation of the cell's Liver X Receptor (LXR) and/or Retinoid X Receptor (RXR).

This invention further provides a method for inhibiting the onset of Alzheimer's disease in a subject comprising administering to the subject a prophylactically effective amount of an agent that, when in contact with a neuronal cell, causes activation of the cell's Liver X Receptor (LXR) and/or Retinoid X Receptor (RXR).

This invention further provides a method for treating a subject afflicted with a disorder characterized by abnormally high Aβ peptide production in the subject's neuronal cells comprising administering to the subject a therapeutically effective amount of an agent that, when in contact with a neuronal cell, causes activation of the cell's Liver X Receptor (LXR) and/or Retinoid X Receptor (RXR).

This invention further provides a method for inhibiting the onset of a disorder in a subject characterized by abnormally high Aβ peptide production in the subject's neuronal cells comprising administering to the subject a prophylactically effective amount of an agent that, when in contact with a neuronal cell, causes activation of the cell's Liver X Receptor (LXR) and/or Retinoid X Receptor (RXR).

This invention further provides an article of manufacture comprising (a) a packaging material having therein an agent that, when in contact with a neuronal cell, causes activation of the cell's Liver X Receptor (LXR) and/or Retinoid X Receptor (RXR), and (b) a label indicating that the agent is intended for use in treating a subject afflicted with Alzheimer's disease.

Finally, this invention provides an article of manufacture comprising (a) a packaging material having therein an agent that, when in contact with a neuronal cell, causes activation of the cell's Liver X Receptor (LXR) and/or Retinoid X Receptor (RXR), and (b) a label indicating that the agent is intended for use in inhibiting the onset of Alzheimer's disease in a subject.

### BRIEF DESCRIPTION OF THE FIGURES

FIGS. 1A-1C: LXR activation reduces Aβ secretion from Neuro2a-APP_{Sw} cells. Neuro2a-APP_{Sw} cells were treated with LXR activators (TO or 22(R)-hydroxycholesterol) and in some cases also with RXR activator (9-cis-retinoic acid). Secreted Aβ (sAβ) was detected in medium by immunoprecipitation and immunoblotting. Cellular APP (cAPP) levels were measured by immunoblotting cell lysates. Western blots were scanned and quantified by ImageQuant. The bar graphs show the combined results (means±S.E.) from at least three independent experiments. A, cells were treated with increasing amounts of TO. B, cells were treated with mock transfection (control) or 1 μM TO, μM 9-cis-RA. C, cells were treated with mock transfection or 5 μM 22(R)-hydroxycholesterol (22(R)OHchol), 10 μM 9-cis-RA. #, p<0.05 compared with control. *, p<0.01 compared with control.

FIG. 2: LXR/RXR activation reduces Aβ40 and Aβ42 in medium as determined by enzyme-linked immunosorbent assay. The cells were treated as described in the legend for FIG. 1B. Filled bar, mock transfected control; hatched bar, treated with 1 μM TO, 1 μM 9-cis-RA. The data are the means±S.E., normalized to control value (control value=1; n=4). *, p<0.01 compared with control; #, p<0.05 compared with control.

FIGS. 3A-3C: ABCA1 overexpression inhibits Aβ secretion. A, ABCA1 protein in Neuro2a cells after LXR activation (top panel) or transient transfection (bottom panel), showing similar expression levels to actin. B and C, Aβ peptide in medium was determined by immunoprecipitation and immunoblotting; cellular APP (cAPP) was determined by immunoblotting cell lysates. The bar graphs show combined data from three or more independent experiments. *, p<0.01 compared with mock (mk) control. B, Neuro2a-APP_{Sw} cells were transfected with either mock control plasmid or ABCA1 cDNA. ApoA-I (AI) was added for 6 h where indicated. C, Neuro2a-APP_{Sw} cells were transfected with empty vector (control) or vector containing ABCA1 cDNA or ABCA1 with a mutation in the ATP-binding cassette (Walker motif mutation). Filled bar, no apolipoprotein added; hatched bar, apoA-I added.

FIG. 4: The effect of apoE isoforms on Aβ secretion. Neuro2a-APP_{Sw} cells were transfected with empty vector (control) or ABCA1 vector, and apoE isoforms were added during the last 6 h of the experiment; data are the means±S.E. for five separate experiments conducted in duplicate or triplicate. *, p<0.01 compared with mock transfected; #, p<0.05 compared with ABCA1 transfected without apolipoprotein.

FIGS. 5A-5B: ABCA1 overexpression decreases β-cleavage of APP_{Sw} and γ-cleavage of the 99-amino acid C-terminal fragment of APP. A, Neuro2a-APP_{Sw} cells were transfected with either empty vector or vector containing ABCA1 cDNA. The β-cleavage product C99 was detected by immunoprecipitation of cell lysates with antibody 4G8 followed by immunoblotting with 6E10. Cellular APP (cAPP) level was measured by immunoblotting cell lysates. The data are the means±S.E. for five separate experiments; *, p<0.01 compared with mock (mk) transfected. B, Neuro2a cells were transfected with vector containing either C99 Myc alone or C99 Myc with ABCA1. Aβ was detected as in FIG. 3. Cellular C99 was detected by immunoprecipitation with anti-Myc antibody followed by Western blot with 6E10. The data are shown for three separate experiments conducted in duplicate. #, p<0.05 compared with C99 transfected alone.

FIGS. 6A-6B: SCD overexpression inhibits Aβ secretion in Neuro2A cells. A, SCD protein in Neuro2A cells before and after LXR activation, as determined by Western blotting using an antibody that recognizes both forms of SCD (SCD1 and SCD2). B, Neuro2A cells were transfected with wild type APP or APP with SCD expression plasmid. Aβ secretion was measured during 4 h of incubation. The data are shown for three separate experiments conducted in duplicate. *, p<0.01 compared with APP transfected alone.

FIGS. 7A-7B: SCD overexpression inhibits Aβ secretion from APP and C99 in 293 cells. A, 293 cells were transfected with APP (wild type) or APP with SCD expressing plasmid. B, 293 cells were transfected with C99 or C99 with SCD expressing plasmid. Aβ expression was measured during 72 h of incubation. *, p<0.01 compared with C99 transfected alone.

### DETAILED DESCRIPTION OF THE INVENTION

Definitions

“APP” is used herein to mean “amyloid precursor protein.”

“ABCA1” is used herein to mean “ATP-binding cassette transporter A1”, and is also referred to in the art as “ABC1”.

“Activate”, when used in connection with a receptor, means to change the receptor's conformation so as to promote transcriptional activity.

“Administering” may be effected or performed using any of the methods known to one skilled in the art. These methods include, for example, intralesional, intramuscular, subcutaneous, intravenous, intraperitoneal, liposome-mediated, transmucosal, intestinal, topical, nasal, oral, anal, ocular and otic means of delivery.

“Agent” shall mean any chemical entity, including, without limitation, a glycomer, a protein, an antibody, a lectin, a nucleic acid, a small molecule, and any combination thereof.

To “cause activation” of a receptor means to activate the receptor either directly (i.e., via direct contact with the receptor) or indirectly (i.e., not via direct contact with the receptor).

“Inhibiting” the onset of a disorder shall mean either lessening the likelihood of the disorder's onset, or preventing the onset of the disorder entirely. In the preferred embodiment, inhibiting the onset of a disorder means preventing its onset entirely.

“LXR” is used herein to mean “liver X receptors.”

“Prophylactically effective amount” means an amount sufficient to prevent, or reduce the likelihood of, the onset of a disorder or a complication associated with a disorder in a subject.

“RXR” is used herein to mean “retinoid X receptors.”

“Subject” shall mean any organism including, without limitation, a mammal such as a mouse, a rat, a dog, a guinea pig, a ferret, a rabbit and a primate. In the preferred embodiment, the subject is a human being.

“Therapeutically effective amount” means an amount sufficient to treat a subject afflicted with a disorder or a complication associated with a disorder. The therapeutically effective amount will vary with the subject being treated, the condition to be treated, the agent delivered and the route of delivery. A person of ordinary skill in the art can perform routine titration experiments to determine such an amount. Depending upon the agent delivered, the therapeutically effective amount of agent can be delivered continuously, such as by continuous pump, or at periodic intervals (for example, on one or more separate occasions). Desired time intervals of multiple amounts of a particular agent can be determined without undue experimentation by one skilled in the art.

“Treating” a disorder means slowing, stopping or reversing the progression of the disorder, and/or ameliorating symptoms associated with a disorder.

### EMBODIMENTS OF THE INVENTION

This invention provides a method for decreasing the amount of Aβ peptide produced by a neuronal cell comprising contacting the cell with an agent that, when in contact with the cell causes activation of the cell's Liver X Receptor (LXR) and/or Retinoid X Receptor (RXR). In one embodiment the agent activates Liver X Receptor (LXR). In another embodiment, the agent activates Retinoid X Receptor (RXR). In another embodiment, the agent is 22(R) hydroxycholesterol. In another embodiment, the agent is 9-cis retinoic acid. In another embodiment, the agent is TO9013.

This invention further provides a method for treating a subject afflicted with Alzheimer's disease comprising administering to the subject a therapeutically effective amount of an agent that, when in contact with a neuronal cell, causes activation of the cell's Liver X Receptor (LXR) and/or Retinoid X Receptor (RXR).

This invention further provides a method for inhibiting the onset of Alzheimer's disease in a subject comprising administering to the subject a prophylactically effective amount of an agent that, when in contact with a neuronal cell, causes activation of the cell's Liver X Receptor (LXR) and/or Retinoid X Receptor (RXR). In one embodiment, the agent activates Liver X Receptor (LXR). In another embodiment, the agent activates Retinoid X Receptor (RXR). In another embodiment, the agent is 22(R) hydroxycholesterol. In another embodiment, the agent is 9-cis retinoic acid. In another embodiment, the agent is TO9013.

This invention further provides a method for treating a subject afflicted with a disorder characterized by abnormally high Aβ peptide production in the subject's neuronal cells comprising administering to the subject a therapeutically effective amount of an agent that, when in contact with a neuronal cell, causes activation of the cell's Liver X Receptor (LXR) and/or Retinoid X Receptor (RXR).

This invention further provides a method for inhibiting the onset of a disorder in a subject characterized by abnormally high Aβ peptide production in the subject's neuronal cells comprising administering to the subject a prophylactically effective amount of an agent that, when in contact with a neuronal cell, causes activation of the cell's Liver X Receptor (LXR) and/or Retinoid X Receptor (RXR). In one embodiment, the agent activates Liver X Receptor (LXR). In another embodiment, the agent activates Retinoid X Receptor (RXR). In another embodiment, the agent is 22(R) hydroxycholesterol. In another embodiment, the agent is 9-cis retinoic acid. In another embodiment, the agent is TO9013.

A person of ordinary skill in the art can perform routine titration experiments to determine therapeutically and prophylactically effective amounts. In one embodiment, the amount is from about 1 mg of agent/subject to about 1 g of agent/subject per dosing. In another embodiment, the amount is from about 10 mg of agent/subject to 500 mg of agent/subject. In a further embodiment, the amount is from about 50 mg of agent/subject to 200 mg of agent/subject. In a further embodiment, the amount is about 100 mg of agent/subject. In still a further embodiment, the amount is selected from 50 mg of agent/subject, 100 mg of agent/subject, 150 mg of agent/subject, 200 mg of agent/subject, 250 mg of agent/subject, 300 mg of agent/subject, 400 mg of agent/subject and 500 mg of agent/subject. Depending upon the agent delivered, the amount of agent can be delivered continuously, such as by continuous pump, or at periodic intervals (for example, on one or more separate occasions). Desired time intervals of multiple amounts of a particular agent can be determined without undue experimentation by one skilled in the art.

This invention further provides an article of manufacture comprising (a) a packaging material having therein an agent that, when in contact with a neuronal cell, causes activation of the cell's Liver X Receptor (LXR) and/or Retinoid X Receptor (RXR), and (b) a label indicating that the agent is intended for use in treating a subject afflicted with Alzheimer's disease. In one embodiment, the agent activates Liver X Receptor (LXR). In another embodiment, the agent activates Retinoid X Receptor (RXR). In another embodiment, the agent is 22(R) hydroxycholesterol. In another embodiment, the agent is 9-cis retinoic acid. In another embodiment, the agent is TO9013. In the preferred embodiment, the subject is a human.

This invention further provides an article of manufacture comprising (a) a packaging material having therein an agent that, when in contact with a neuronal cell, causes activation of the cell's Liver X Receptor (LXR) and/or Retinoid X Receptor (RXR), and (b) a label indicating that the agent is intended for use in inhibiting the onset of Alzheimer's disease in a subject. In one embodiment, the agent activates Liver X Receptor (LXR). In another embodiment, the agent activates Retinoid X Receptor (RXR). In another embodiment, the agent is 22(R) hydroxycholesterol. In another embodiment, the agent is 9-cis retinoic acid. In another embodiment, the agent is TO9013. In the preferred embodiment, the subject is a human.

This invention will be better understood from the Experimental Details which follow. However, one skilled in the art will readily appreciate that the specific methods and results discussed are merely illustrative of the invention as described more fully in the claims which follow thereafter.

Experimental Details

Synopsis

A hallmark of Alzheimer's disease is the deposition of plaques of Aβ in the brain. Aβ is thought to be formed from APP in cholesterol-enriched membrane rafts, and cellular cholesterol depletion decreases Aβ formation. LXRs play a key role in regulating genes that control cellular cholesterol efflux and membrane composition and are widely expressed in cells of the central nervous system. It is shown that treatment of APP-expressing cells with LXR activators reduces the formation of Aβ. LXR activation results in increased levels of ABCA1 and stearoyl CoA desaturase, and expression of these genes individually decreases formation of Aβ. Expression of ABCA1 leads to both decreased β-cleavage product of APP_{Sw} (i.e. C99 peptide) and reduced γ-secretase-cleavage of C99 peptide. Remarkably, these effects of ABCA1 on APP processing are independent of cellular lipid efflux. LXR and ABCA1-induced changes in membrane lipid organization have favorable effects on processing of APP, indicating a new approach to the treatment of Alzheimer's disease.

Materials and Methods

Cell Culture

Mouse neuroblastoma Neuro2a cells stably expressing Swedish APP695 were previously described (12). Neuro2a-APP_{Sw} cells were grown in Dulbecco's modified Eagle's medium (DMEM)/OptiMEM supplemented with 5% fetal bovine serum at 37° C. in a humidified 5% CO₂ incubator. Tissue culture reagents were from Invitrogen. Transient and stable transfections were performed with LipofectAMINE 2000 (Invitrogen). 22(R)-Hydroxycholesterol and the synthetic LXR activator TO901317 were purchased from Sigma. 9-cis-Retinoic acid (9-cis-RA) was from Biomol, apoA-I was from Biodesign, and the apoE isoforms were from Calbiochem.

Immunoblots

The cells were lysed in buffer (10 mM Tris, pH 7.5, 150 mM NaCl, 1% Triton X-100, 0.25% Nonidet P-40, and 2 mM EDTA) supplemented with protease inhibitor mixture (Roche Applied Science). Postnuclear lysates were collected by spinning the lysed cells at 8000×g for 5 min. Postnuclear lysates were fractionated in 4-15% SDS-polyacrylamide gel electrophoresis and transferred to 0.2 μm nitrocellulose membranes (Bio-Rad). Polyclonal anti-ABCA1 antibody was purchased from Novus (Littleton, Colo.). Monoclonal anti-actin antibody was purchased from Sigma. Polyclonal anti-SCD antibodies were raised in rabbits (13). Cellular APP was detected by monoclonal antibody 22C11 (Chemicon).

Detection of Secreted Aβ

In experiments with LXR activation, the cells were first induced with activators for 24 h in DMEM, 5% lipoprotein-deficient serum. The cells were then incubated in fresh medium for 4 h for Aβ measurements. In experiments with ABCA1 transient transfections, 24 h after transfection, the cells were incubated with DMEM, 1% lipoprotein-deficient serum with or without apolipoproteins for 6 h for Aβ measurements. After the indicated treatments, conditioned medium was collected on ice and centrifuged at 6000×g for 10 min to remove cell debris. Immunoprecipitation of Aβ or C99 was performed with monoclonal antibody 4G8 (Signet) and protein A/G-conjugated agarose (Santa Cruz). Aβ and C99 were then extracted in NuPAGE sample buffer (Invitrogen) and fractionated in 4-12% NuPAGE Bis-Tris Gel (Invitrogen). Fractionated proteins were then transferred to polyvinylidene difluoride membrane (Bio-Rad) and blotted with monoclonal antibody 6E10 (Signet) after boiling. The immunoblots were developed using the ECL system (Pierce) scanned and quantified by ImageQuant (Molecular Dynamics). Quantitation of Aβ40 and Aβ42 was performed using a commercial Aβ enzyme-linked immunosorbent assay kit. (BIOSOURCE).

Cellular Cholesterol Efflux

The efflux experiments were performed as described (13). Briefly, Neuro2a cells were labeled with 1 μCi/ml [1.2-³H(N)]-cholesterol (PerkinElmer Life Sciences) in DMEM containing 5 mM methyl-β-cyclodextrin:cholesterol at a molar ratio of 8:1 for 15 min at 37° C. After washing, the cells were equilibrated in DMEM, 0.2% bovine serum albumin for 30 min and then used for efflux experiments. The cells were incubated with 10 μg/ml purified human apoA-I or apoEs in DMEM, 1% lipoprotein-deficient serum for 6 h, and the medium was collected and centrifuged at 6000×g for 10 min to remove cell debris and cholesterol crystals. The cells were lysed in 0.1 M sodium hydroxide, 0.1% SDS, and radioactivity was determined by liquid scintillation counting. Efflux was expressed as the percentage of radioactivity in the medium relative to the total radioactivity in cells and medium.

Statistical Analysis

The significance of differences between groups was assessed by Student's t test.

Results

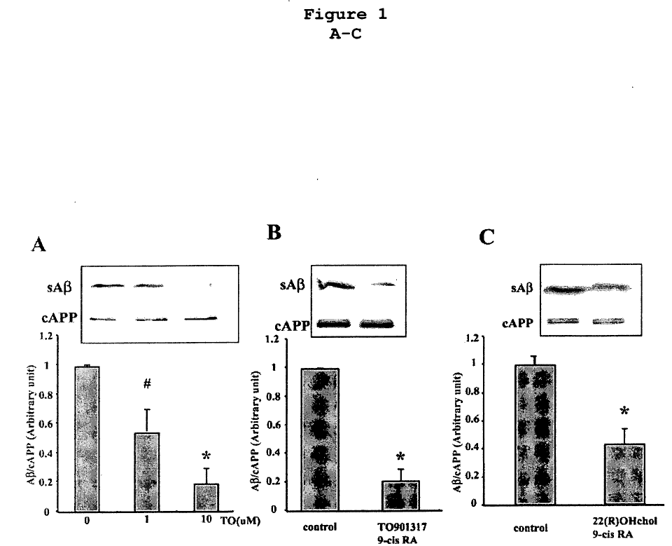

To evaluate the effects of LXR activators on APP processing, neuron-derived Neuro2a cells, stably transfected with human APP_{Sw} containing a mutation that increases the formation of total Aβ (the Swedish mutation), were employed (14). The cells were treated with increasing doses of the synthetic LXRα/β activator TO901317 (“TO”), and Aβ secretion into medium was measured by immunoprecipitation and Western blotting (FIG. 1A). This revealed a decrease in the secretion of Aβ, with an approximate 50% reduction at 1 μM TO. The amount of the soluble form of APP (APPsα) in medium was not significantly changed by administration of the LXR activator (not shown). LXR acts in a heterodimeric complex with retinoid X receptor (RXR), and the response of genes to LXR activators is increased in the presence of RXR activators, such as 9-cis-retinoic acid (15). When cells were treated with 1 μM TO901317 plus 1 μM 9-cis-retinoic acid, there was a further reduction in Aβ secretion to 20% of control (FIG. 1B). Aβ formation was reduced when the cells were treated with the natural LXR activator, 22(R)-OH cholesterol, together with 9-cis-retinoic acid (FIG. 1C). LXR/RXR activation also decreased the secretion of Aβ formed from endogenous APP in 293 cells (data not shown), indicating that these effects were not dependent on overexpression of mutant APP.

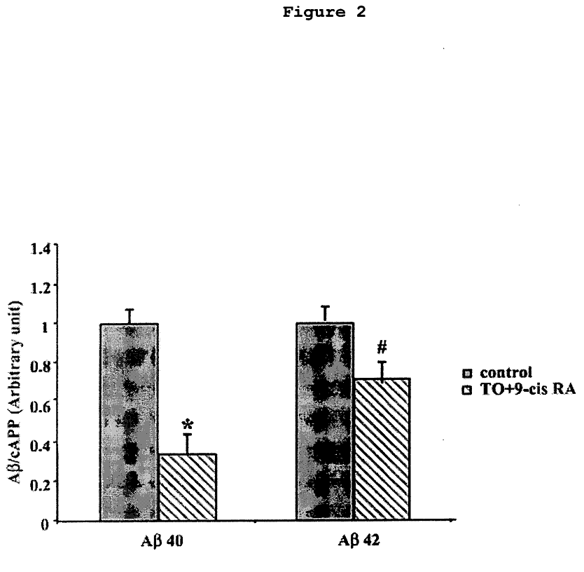

Aβ is secreted in several different forms. Although Aβ40 (40 amino acids) is the predominant species, Aβ42 (42 amino acids) is a minor, but more amyloidogenic form. Whereas Aβ42 is formed predominantly in the endoplasmic reticulum and transgolgi, Aβ40 is made in plasma membrane, endocytic compartments, and trans-Golgi (16, 17). Measurement of both forms of Aβ in medium of Neuro2a cells by enzyme-linked immunosorbent assay revealed that LXR/RXR activation (1 μM TO, 1 μM 9-cis-RA) decreased the secretion of Aβ40 by about 70%, whereas Aβ42 was more moderately reduced (FIG. 2). Because the concentration of Aβ40 in medium was ˜8-fold more than that of Aβ42, the marked decrease in Aβ signal in FIG. 1 primarily reflects a reduction in Aβ40.

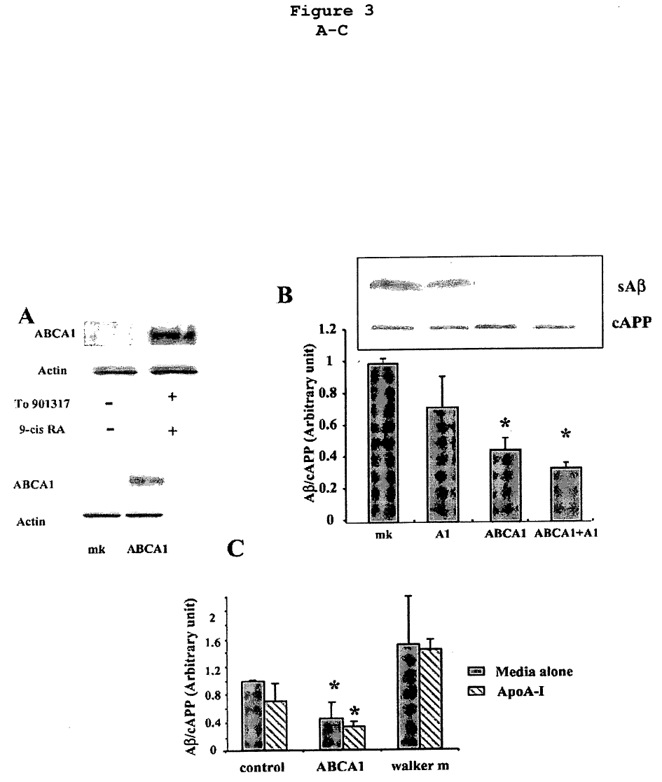

Neuro2a cells treated with LXR/RXR activators (1 μM TO and 1 μM 9-cis-RA) showed a marked induction of ABCA1 protein (FIG. 3A). To determine whether induction of ABCA1 might be responsible for decreased secretion of Aβ, Neuro2a cells were transfected with ABCA1 and incubated with or without apoA-I. Transfection of ABCA1 resulted in levels of ABCA1 protein comparable with that induced by LXR/RXR activators (FIG. 3A). Expression of ABCA1 decreased the formation of Aβ without affecting cellular APP levels (FIG. 3B). Surprisingly, the major effect of ABCA1 expression was observed without the addition of the extracellular acceptor apoA-I, and there was only a slight further decrease in Aβ when apoA-I was added to medium (FIG. 3B). Measurement of cellular cholesterol efflux showed a small but significant increase in efflux (1.0±0.1% of total cellular cholesterol) with expression of ABCA1 and the addition of apoA-I. Similarly, there was an increase in efflux of cellular phospholipids (1.9% of total cellular choline-labeled lipids). However, expression of ABCA1 alone did not increase cholesterol or phospholipid efflux compared with nontransfected controls. Cellular cholesterol mass was also measured. Under these experimental conditions, there was no detectable cholesteryl ester in cells, either with or without expression of ABCA1.

As a control for possible nonspecific effects of ABCA1 expression unrelated to functional activity, Neuro2a cells were transfected with a mutant form of ABCA1 (Walker motif mutant). This mutant is well expressed on the cell surface but inactive both in lipid efflux and binding of apoA-I (18). When expressed at levels similar to those of wild type ABCA1, the mutant failed to alter Aβ secretion by Neuro2a cells (FIG. 3C), indicating that the effects of ABCA1 expression are related to the activity of the transporter, even though they do not require lipid efflux.

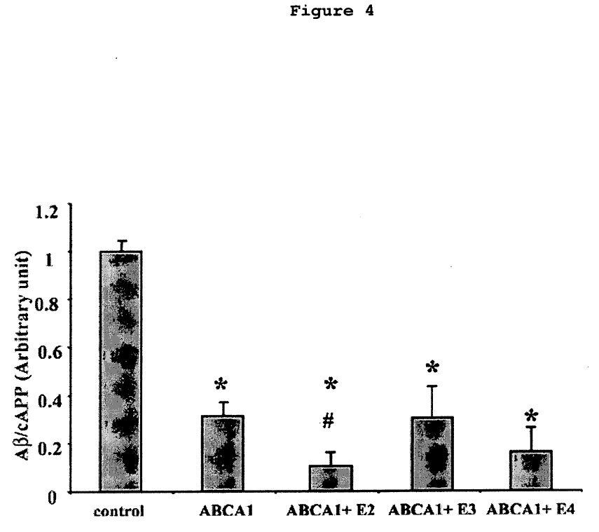

Because apoE is a major apolipoprotein in the central nervous system and the apoE4 isoform is associated with increased risk of AD (19, 20), the effects of apoE on Aβ formation was also examined. Expression of ABCA1 with the addition of apoE also resulted in a profound decrease in Aβ formation (FIG. 4). Again, the major effect was attributable to ABCA1 expression alone. The addition of apoE2 resulted in a small but significant further decrease in Aβ formation, whereas apoE3 and apoE4 did not produce significant further reductions in Aβ secretion. The addition of apoE isoforms without ABCA1 expression did not affect Aβ secretion (not shown).

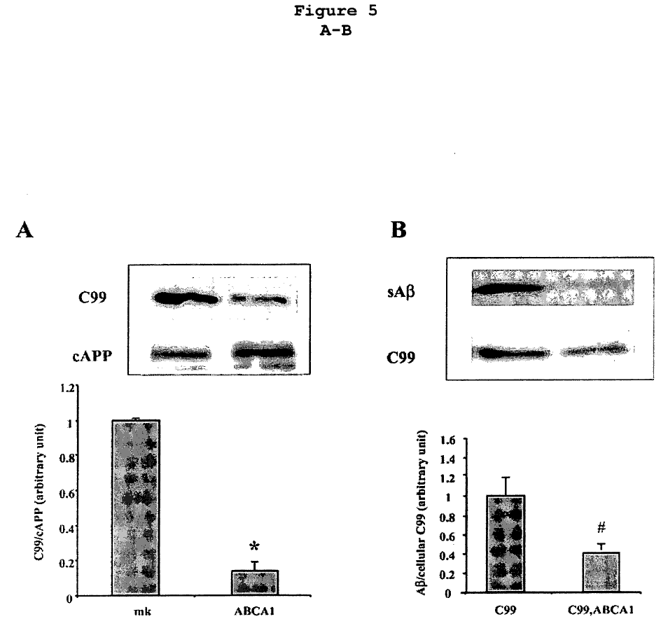

Both β and γ-cleavage of APP are required for the generation of Aβ. A series of experiments were carried out to determine which step is affected by ABCA1 overexpression. To measure β-cleavage, the β-secretase cleavage product was immunoprecipitated (C99 ) from cell lysates. There was an 85% decrease of cellular C99 in Neuro2a-APP_{Sw} cells transiently transfected with ABCA1 compared with mock transfected control (FIG. 5A). γ-Secretase mediates the final step in Aβ generation (1). To assess the direct effects of ABCA1 expression on γ-secretase cleavage of APP, Neuro2a cells were transfected with constructs encoding C-terminal APP fragments (C99) containing a Myc epitope tag. ABCA1 expression was found to decrease the cleavage of C99 peptide, indicating a decrease in γ-secretase processing (FIG. 5B). The addition of apoA-I did not provide a further decrease in Aβ generation (not shown).

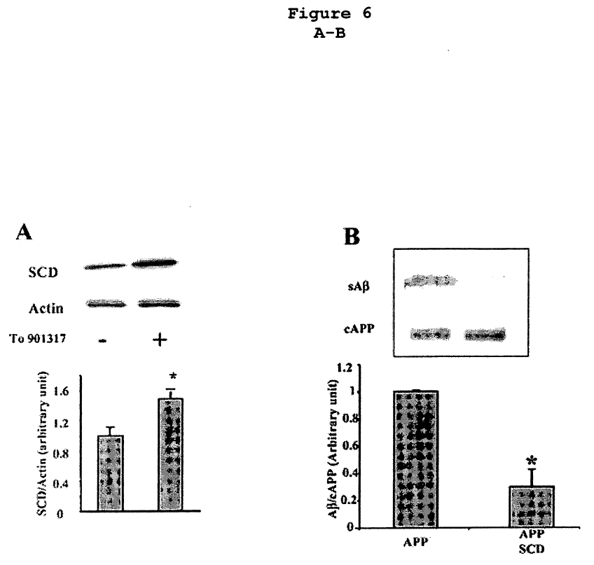

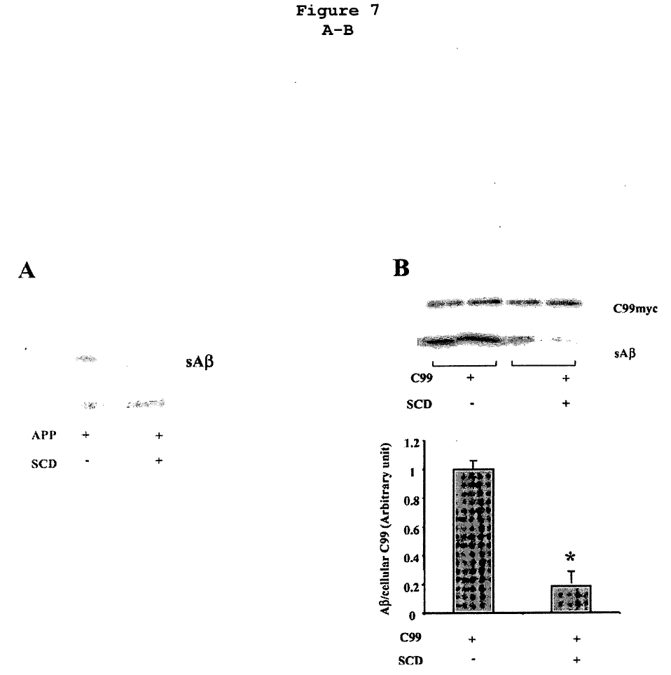

In addition to targeting genes involved in cellular cholesterol efflux and transport, LXRs also activate synthesis of mono-unsaturated fatty acids (21). SCD is a key LXR target gene that catalyzes the conversion of stearoylCoA to oleoylCoA and increases the content of mono-unsaturated fatty acids in cell membrane phospholipids (22). It was recently shown that SCD activity decreases the amount of liquid ordered domains in the plasma membrane (13). There are two forms of SCD, both LXR targets, with similar catalytic activity and cellular effects (22). Using an antibody that recognizes both forms of SCD, it was shown that treatment of Neuro2a cells with LXR activators resulted in a modest 1.6-fold increase in SCD protein (FIG. 6A). Transient transfection of SCD in Neuro2a cells resulted in a decrease in Aβ secretion into medium (FIG. 6B). Because Neuro2a cells have high basal levels of SCD activity, similar experiments in 293 cells that have much lower basal SCD expression were also carried out (13). Transient overexpression of SCD resulted in an increase in APPsα formation (not shown) and a profound decrease in Aβ generation (FIG. 7A) that was associated with a marked decrease in γ-secretase cleavage of the C99 peptide (FIG. 7B).

Discussion

The idea that changes in cellular lipid metabolism might favorably influence APP processing has attracted considerable attention (23). Cellular cholesterol depletion, based on treatment with cyclodextrins or statins, reduces membrane rafts, increases α-cleavage of APP, and reduces β-cleavage, leading to less Aβ secretion (24). One explanation for these observations is decreased partitioning or trafficking of APP into cholesterol-enriched membrane domains where the β-cleavage enzyme resides (23, 25). In contrast, in this study, LXR activators and ABCA1 expression were observed to decrease Aβ secretion in the absence of cellular lipid efflux, and this was not accompanied by changes in α-cleavage and was mediated in part by a decrease in γ-secretase cleavage of the C-terminal 99 amino acid peptide of APP. The results suggest that ABCA1-mediated translocation of membrane cholesterol leads to a decrease in γ-secretase cleavage. This suggests either sensitivity of γ-secretase activity to membrane environment or altered trafficking of C99 to the site of γ-cleavage and represents a new mechanism to link alteration in membrane lipid organization and Aβ production.

These studies were undertaken to test whether LXR activation would increase lipid efflux and thereby decrease Aβ generation. However, LXR activators decreased Aβ formation in the absence of extracellular acceptors (FIG. 1), and the addition of apoA-I or any of the three isoforms of apoE resulted in only minor further changes in Aβ (FIGS. 3 and 4). Serving as a control for nonspecific effects related to ABCA1 expression, the Walker motif mutant of ABCA1 failed to decrease Aβ formation (FIG. 3C). These findings indicate that the decrease in Aβ is related to an intrinsic cellular activity of ABCA1. ABCA1 probably acts as a lipid translocase at the plasma membrane (26) and causes changes in plasma membrane morphology (27). Recently, Vaughan and Oram (28) showed that ABCA1 expression increases cell surface cholesterol oxidase-accessible domains, indicating a redistribution of cholesterol toward the outer membrane, independent of extracellular lipid acceptors. Decreased Aβ formation might result from an ABCA1-induced redistribution of membrane cholesterol either at the plasma membrane or in the Golgi or endocytic compartments.

Although ABCA1-mediated lipid translocation could affect raft organization, it seems unlikely that an alteration in plasma membrane rafts fully accounts for these findings. ABCA1 appears to be localized in and to induce cholesterol efflux from nonraft membrane regions (29), and ABCA1 expression did not alter the distribution or amount of plasma membrane liquid ordered regions in 293 cells. In contrast, SCD activity decreases membrane liquid ordered regions (13), and was associated with increased α-cleavage of APP, similar to the effects of cholesterol depletion. However, changes in SCD protein levels in Neuro2a cells were modest and unlikely to account for a major part of the effect of LXR activators.

Both ABCA1 and SCD expression were associated with a decrease in γ-secretase cleavage of the C-terminal 99 amino acids of APP (FIGS. 5B and 7). In the case of APP_{Sw}, ABCA1 decreased β-cleavage by 85% (FIG. 5A), but the overall decrease in Aβ secretion was only around 60% (FIG. 3B), which is comparable with the effect of ABCA1 on γ-cleavage (FIG. 5B). This suggests that under these conditions γ-cleavage is the rate-limiting step in Aβ secretion. These findings suggest that this key enzyme of Aβ formation is highly sensitive to its membrane lipid environment, possibly reflecting the fact that γ-secretase mediates an intramembranous cleavage of APP. Alterations in membrane fluidity caused by lipid translocation or increased content of mono-unsaturated phospholipids could lead to a decrease in γ-secretase activity. Although the cellular compartmentalization of γ-secretase activity is not completely understood (1), several reports suggest that Aβ formation mainly occurs in the endocytic compartments (30) where components of γ-secretase, including presenilins, are shown to be present (31). An alternative interpretation of these results is that induction of ABCA1 activity results in altered cellular trafficking of APP or the C-terminal fragment of APP, providing less substrate for γ-secretase cleavage. The finding that cellular cholesterol depletion leads to less Aβ secretion has fostered the idea that statins could be useful in the treatment of AD. Epidemiological studies have suggested that statin therapy is associated with decreased prevalence of AD (32, 33). However, brain cholesterol is derived by local synthesis (not from plasma low density lipoprotein), and statins would have to be present in the brain at high levels to alter neuronal lipid metabolism. Although human studies show an association between statin treatment and decreased prevalence of AD, such associations can reflect the influence of confounding factors, as appears to be the case for statins and bone disease (34). A recent placebo-controlled prospective trial of statin therapy in the elderly failed to show any improvements in cognitive function (35).

Inhibition of cholesterol esterification by acylCoA:cholesterol acyl transferase inhibitors has also been proposed to favorably affect processing of APP (36). Because cholesteryl esters have minor solubility in membranes and are thought to be present in cells as inert lipid droplets, it is unlikely that these effects are related to cholesteryl ester accumulation. One possibility suggested by these studies is that acylCoA:cholesterol acyl transferase inhibition leads to accumulation of cellular free sterol and conversion to LXR ligands via endogenous oxysterol synthesizing enzymes such as 24-cholesterol hydroxylase (37). Cholesteryl esters were not stored in cells in appreciable amounts under the conditions of these experiments, and changes in cellular acylCoA:cholesterol acyl transferase activity are thus unlikely to account for the findings.

These findings, along with another recent report (38), suggest that LXR activators currently being developed for the treatment of atherosclerosis might have therapeutic efficacy in Alzheimer's disease. LXR/RXR activation markedly decreased Aβ40 but had less effect on Aβ42 secretion. Although Aβ42 is thought to be more amyloidogenic than Aβ40, much more Aβ40 is secreted by neurons, and both forms are found in amyloid plaques (2). Therefore, controlling the predominant Aβ40 secretion could be beneficial. These results differ from another report, where a 65% increase in Aβ42 and no significant change in Aβ40 was found in Neuro2a cells treated with TO and 9-cis-retinoic acid (10 μM) (39). The reasons for this discrepancy are not clear. However, these findings substantially agree with another report (38), which appeared while these findings were under review. The subject results extend these latter studies by the direct demonstration that ABCA1 and SCD expression decreases Aβ formation and that these effects are observed without lipid efflux from cells and involve a decrease in γ-secretase cleavage.

In contrast with the more general approach of cholesterol biosynthesis inhibition, LXR activation may directly regulate genes that favorably modulate plasma membrane composition and structure in the brain (8). Tangier disease patients have not been reported with premature dementia, suggesting that ABCA1 may not have an essential role in protecting against AD. However, this does not rule out the possibility that increased expression of ABCA1 has a protective role, just as it does in atherosclerosis (40).

### REFERENCES

1. Esler, W. P. and Wolfe. M. S. (2001) Science 293, 1449-1454.
2. Hardy, J. and Selkoe, D. J., (2002) Science 297, 353-356.
3. Simons, K. and Ehehalt, R., (2002) J. Clin. Invest. 110, 597-603.
4. Simons, M. et al., (1998) Proc. Natl. Acad. Sci. U.S.A. 95, 6460-6464.
5. Parkin, E. T., et al., (1998) J. Neurochem. 72, 1534, 1543.
6. Horton, J. D., et al., (2002) J. Clin. Invest. 109, 1125-1131.
7. Tall, A. R., et al., (2000) Nat. Med. 6, 1104-1105.
8. Wang, L., et al., (2002) Proc. Natl. Acad. Sci. U.S.A. 99, 13878-13883.
9. Costet, P., et al., (2000) J. Biol. Chem. 275, 28240-28245.
10. Remaley, A. T., et al., (2001) Biochem. Biophys. Res. Commun. 280, 818-823.
11. Whitney, K. D., et al., (2002) Mol. Endocrinol. 16, 1378-1385.
12. Thinakaran, G., et al., (1996) J. Biol. Chem. 271, 9390-9397.
13. Sun, Y., et al., (2003) J. Biol. Chem. 278, 5813-5820.
14. Haass, C., et al., (1995) Nat. Med. 1, 1291-1296.
15. Peet, D. J., et al., (1998) Curr. Opin. Genet. Dev. 8, 571-575.
16. Hartmann, T., et al., (1997) Nat. Med. 3, 1016-1020.
17. Greenfield, J. P., et al., (1999) Proc. Natl. Acad. Sci. U.S.A. 96, 742-747.
18. Wang, N., et al., (2001) J. Biol. Chem. 276, 23742-23747.
19. Corder, E. H., et al., (1993) Science 261, 921-923.
20. Schmechel, D. E., et al., (1993) Proc. Natl. Acad. Sci. U.S.A. 90, 9649-9653.
21. Repa, J. J., et al., (2000) Genes Dev. 14, 2819-2930.
22. Ntambi, J. M., (1999) J. Lipid. Res. 40, 1549-1558.
23. Wolozin, B., (2001) Proc. Natl. Acad. Sci. U.S.A. 98, 5371-5373.
24. Kojro, E., et al., (2001) Proc. Natl. Acad. Sci. U.S.A. 98, 5815-5820.
25. Ehehalt, et al., (2003) J. Cell Biol. 160, 113-123.
26. Hamon, Y., et al., (2000) Nat. Cell Biol. 2, 399-406.
27. Wang, N., et al., (2000) J. Biol. Chem. 275, 33053-33058.
28. Vaughan, A. M. and Oram, J. F., (2003) J. Lipid. Res. 44(7), 1373-1380.
29. Mendez, A. J., et al., (2001) J. Biol. Chem. 276, 3158-3166.
30. Perez, R. G., et al., (1999) J. Biol. Chem. 274, 18851-18856.
31. Kaether, C., et al., (2002) J. Cell. Biol. 158, 551-561.
32. Wolozin, B., et al., (2000) Arch. Neurol. 57, 1439-1443.
33. Jick, H., et al., (2000) Lancet 356, 1627-1631.
34. Coons, J. C., et al., (2002) Ann. Pharmacopher. 36, 326-330.
35. Sheperd, J., et al., (2002) Lancet, 360, 1623-1630.
36. Puglielli, L., et al., (2001) Nat. Cell Biol. 3, 905-912.
37. Lutjohann, D., et al., (1996) Proc. Natl. Acad. Sci. U.S.A. 93, 9799-9804.
38. Koldamova, R. P., et al., (2003) J. Biol. Chem. 278, 13244-13256.
39. Fukumoto, H., et al., (2002) J. Biol. Chem. 277, 48508-48513.
40. Singaraja, R. R., et al., (2002) J. Clin. Invest. 110, 35-42.

1. A method for decreasing the amount of Aβ peptide produced by a neuronal cell comprising contacting the cell with an agent that, when in contact with the cell, causes activation of the cell's Liver X Receptor (LXR) and/or Retinoid X Receptor (RXR).

2. The method of claim 1, wherein the agent activates Liver X Receptor (LXR).

3. The method of claim 1, wherein the agent activates Retinoid X Receptor (RXR).

4. The method of claim 1, wherein the agent is 22(R) hydroxylcholesterol.

5. The method of claim 1, wherein the agent is 9-cis retinoic acid.

6. The method of claim 1, wherein the agent is TO901317.

7. A method for treating a subject afflicted with Alzheimer's disease comprising administering to the subject a therapeutically effective amount of an agent that, when in contact with a neuronal cell, causes activation of the cell's Liver X Receptor (LXR) and/or Retinoid X Receptor (RXR).

8. A method for inhibiting the onset of Alzheimer's disease in a subject comprising administering to the subject a prophylactically effective amount of an agent that, when in contact with a neuronal cell, causes activation of the cell's Liver X Receptor (LXR) and/or Retinoid X Receptor (RXR).

9. The method of claim 7 or 8, wherein the agent activates Liver X Receptor (LXR).

10. The method of claim 7 or 8, wherein the agent activates Retinoid X Receptor (RXR).

11. The method of claim 7 or 8, wherein the agent is 22(R) hydroxylcholesterol.

12. The method of claim 7 or 8, wherein the agent is 9-cis retinoic acid.

13. The method of claim 7 or 8, wherein the agent is TO901317.

14. A method for treating a subject afflicted with a disorder characterized by abnormally high Aβ peptide production in the subject's neuronal cells comprising administering to the subject a therapeutically effective amount of an agent that, when in contact with a neuronal cell, causes activation of the cell's Liver X Receptor (LXR) and/or Retinoid X Receptor (RXR).

15. A method for inhibiting the onset of a disorder in a subject characterized by abnormally high Aβ peptide production in the subject's neuronal cells comprising administering to the subject a prophylactically effective amount of an agent that, when in contact with a neuronal cell, causes activation of the cell's Liver X Receptor (LXR) and/or Retinoid X Receptor (RXR).

16. The method of claim 14 or 15, wherein the agent activates Liver X Receptor (LXR).

17. The method of claim 14 or 15, wherein the agent activates Retinoid X Receptor (RXR).

18. The method of claim 14 or 15, wherein the agent is 22(R) hydroxylcholesterol.

19. The method of claim 14 or 15, wherein the agent is 9-cis retinoic acid.

20. The method of claim 14 or 15, wherein the agent is TO901317.

21-28. (canceled)
